(19) United States
(12) Patent Application Publication
(10) Pub. No.: US 2021/0401618 A1
(43) Pub. Date: Dec. 30, 2021

(54) IMPLANTABLE PROBE FOR LOCALIZED COOLING

(71) Applicants: Commissariat à l'Energie Atomique et aux Energies Alternatives, Paris (FR); UNIVERSITE GRENOBLE ALPES, Saint Martin D'Heres (FR); CENTRE HOSPITALIER UNIVERSITAIRE GRENOBLE ALPES, La Tronche (FR)

(72) Inventors: Quentin BORNTRAGER, Bourg Saint Maurice (FR); Claude CHABROL, Grenoble Cedex 9 (FR); Stephan CHABARDES, Saint Martin D'Heres (FR)

(73) Assignees: Commissariat à l'Energie Atomique et aux Energies Alternatives, Paris (FR); UNIVERSITE GRENOBLE ALPES, Saint Martin D'Heres (FR); CENTRE HOSPITALIER UNIVERSITAIRE GRENOBLE ALPES, La Tronche (FR)

(21) Appl. No.: 17/304,787

(22) Filed: Jun. 25, 2021

(30) Foreign Application Priority Data
Jun. 25, 2020 (FR) 20 06641

(51) Int. Cl. A61F 7/12

(52) U.S. Cl. CPC A61F 7/12; A61F 2007/0002; A61F 2007/126

(57) ABSTRACT

A probe intended to be at least partially implanted in a living being, with a view to locally cooling at least one region of the living being, this probe notably including a cooling member present at its distal end, which member is intended to make contact with the region to be cooled, and a thermoelectric cooling module including a cold region making contact with the cooling member.

### TECHNICAL FIELD OF THE INVENTION

The present invention relates to a probe intended to be at least partially implanted into a living being, with a view to cooling, locally, at least one region of the living being.

The invention also relates to a cooling device incorporating said probe.

### PRIOR ART

It is known to cool a region of the brain using a probe having, at its end, a cooling finger.

The objective of these probes is notably to achieve localized cooling of target tissues in the brain with a view to treating diseases such as: epilepsy, depression, OCD, essential tremor, Parkinson's disease, dystonia, obesity, Tourette syndrome, etc.

The target tissues are for example: the GPi, the STN, the thalamus in its entirety, bundles of fibres such as the MFB (median forebrain bundle), the ALIC, the CG 25, the hippocampus, the corpus amygdaloideum and any epileptogenic, deep lesion or structure (dysplasia, polymicrogyria, nodular heterotopia, etc.).

Such probes are notably described in patent U.S. Pat. No. 8,202,308B2, and in patent application US2013/072776A1.

Thus, U.S. Pat. No. 8,202,308B2 provides a probe having a cooling element placed on the exterior of the skull and a heat-transporting device, taking the form of a heat pipe, allowing the cold finger making contact with the brain to be cooled via transportation of heat to the exterior.

For its part, patent application US2013/072776A1 provides a (cortical) areal cooling probe using a Peltier-effect module placed directly at the distal end of the probe at which the cooling finger is found, thus allowing heat loss between this module and the cold finger to be limited. A liquid-based cooling device is employed to remove heat from the distal end of the probe to the exterior of the skull.

In both these solutions, the dissipation of heat is not optimal. In the first document, the heat may be removed directly to the exterior because the cooling module is placed outside of the cranium, but this solution requires heat to be transported from the cold finger, which may give rise to heat loss, and a very good thermal insulation along the probe. In the second document, heat must be removed from the end of the probe to the exterior of the human body using a complex and bulky solution that is incompatible with a long-term implant. In both cases, the thermal efficiencies are not optimal and these prior-art solutions can prove to be complex to implement.

Patent applications WO92/20289 and WO03005797A1 also describe cooling-effect probe architectures.

There is a need for a probe that is long-term implantable at depth and that allows a region of the brain to be cooled locally, this probe being optimized from the thermal point of view and from the point of view of its energy efficiency, and minimizing medical risks during use thereof, i.e. the risks of lesions and/or infections of the patient.

### DISCLOSURE OF THE INVENTION

This need is met via a probe intended to be at least partially implanted into a living being, with a view to locally cooling at least one region of the living being, said probe having a shape that is elongate along a longitudinal axis between a first end, referred to as the proximal end, and a second end, referred to as the distal end, said distal end being intended to make contact with the region to be cooled, said probe having a first assembly comprising:
a cooling member present at its distal end, said member being intended to make contact with the region to be cooled,
a thermoelectric cooling module comprising a cold region making contact with the cooling member and a hot region,
a device for transporting heat having a first portion making contact with the hot region of the thermoelectric cooling module and a second portion extending the first portion toward the proximal end of the probe,
a thermally separating first jacket arranged around the first portion of said heat-transporting device,
a heat-dissipating second jacket arranged in contact with the second portion of said heat-transporting device, said heat-dissipating second jacket having a thermal conductivity higher than that of the thermally separating jacket,
electrical connecting means arranged along the probe with a view to powering the thermoelectric cooling module.

According to one particularity, the first assembly is of unitary construction and has a rigid configuration, and the probe comprises a second assembly made of a more supple material extending said first assembly towards its proximal end.

According to another particularity, the probe comprises electrical connecting means arranged on the one hand in the first assembly and on the other hand in the second assembly.

According to another particularity, the second assembly takes the form of a supple tube made of silicone or polyurethane.

According to another particularity, the heat-transporting device is a heat pipe or a heat-transferring system made of pyrolytic graphite.

According to another particularity, the thermally separating first jacket is made of silica or zirconium.

According to another particularity, the heat-dissipating second jacket is made of sapphire or aluminium nitride.

According to another particularity, the thermoelectric cooling module is a Peltier-effect module having a face called the cold face and a face called the hot face.

According to another particularity, the Peltier-effect module is arranged with the cold face and the hot face parallel to said longitudinal axis.

According to another particularity, the Peltier-effect module is arranged with the cold face and the hot face arranged transversely to said longitudinal axis.

According to another particularity, the probe comprises a temperature probe arranged in contact with the cooling member.

The invention also relates to a cooling device comprising a control unit and a probe that is intended to be at least partially implanted in a living being, with a view to locally cooling at least one region of the living being, said probe being such as defined above.

### BRIEF DESCRIPTION OF THE FIGURES

Other features and advantages will become apparent from the following detailed description, which is given with reference to the appended drawings, in which:

FIG. 1 schematically shows the architecture of the cooling device according to the invention;

FIG. 2 schematically shows the cooling device of the invention, the probe of which is implanted into a skull;

FIG. 3 schematically shows the probe of the invention and illustrates its various thermal functions;

FIG. 4 shows, via a see-through perspective view, the first assembly of the probe of the invention;

FIG. 5A shows, via a cross-sectional view, the cooling device according to the invention;

FIG. 5B shows details A, B and C of FIG. 5A, respectively;

FIGS. 6A and 6B show two examples of embodiments of a cold finger located at the distal end of the probe of the invention;

FIG. 7A to 7C show temperature gradients for various shapes of the cooling element of the probe (cold finger or cooling ring);

FIG. 8 shows the curve of variation in the temperature on the periphery of a cold finger at a temperature of 16° C.,

FIGS. 9A and 9B show temperature gradients on the periphery of a cold finger, the finger being at a temperature of 16° C. and at a temperature of 10° C., respectively;

FIG. 10 shows a first example of an embodiment of a Peltier module employed in the probe of the invention;

FIG. 11 shows a second example of an embodiment of a Peltier module employed in the probe of the invention;

FIGS. 12A to 12B show two examples of an embodiment of the Peltier module able to be employed in the probe of the invention;

FIG. 13 shows a variant embodiment of the end of the probe of the invention;

FIG. 14 shows a variant embodiment of the distal end of the probe;

FIG. 15 shows a graph showing the power required over time to keep the cold finger of the probe at a temperature of 4° C.;

FIG. 16 shows a graph showing the variation in the average surface temperature of the cold finger of the probe over time required to obtain cooling of 0.4 W.

### DETAILED DESCRIPTION OF AT LEAST ONE EMBODIMENT

The objective of this invention is to allow the cooling of a volume of brain at depth. The proposed solution for example allows a cerebral volume located at a distance of about 1 mm around the cold finger to be cooled to 20° C., which is the temperature required to prevent an epileptic fit.

Potential targets are the hippocampus, the nucleus of the thalamus or any region that may contain an epileptogenic or tumour focus. This cooling device must be long-term implantable in a human in order to provide treatment of chronic diseases, tumours, or any other disorder liable to benefit from the effects of a heat treatment.

From a thermodynamic point of view, it is known that any body having a temperature higher than 0 K possesses thermal energy. It is therefore not possible to create cold, only transport heat (i.e. calories) from one region to another. This heat transfer occurs spontaneously from hot to cold and tends to bring thermal energies, and therefore temperatures, into equilibrium. If it is desired to invert the natural direction of the heat transfer by transporting heat from a cold region to a hot region (and therefore cool the cold region and heat the hot region) a thermodynamic system must do work.

In the context of the device of the invention, it is a question of cooling a region of the brain by removing therefrom some of its thermal energy. This implies transporting this energy and releasing it elsewhere.

The principle of the invention aims to use the arterial perfusion of the brain to remove the heat absorbed from the region to be cooled. The probe of the invention respects this principle, while being suitable for the anatomy of patients to be treated. It is notably necessary to take care to limit the risks of mechanical lesions in the brain, and the consequence of potential thermal hotspots.

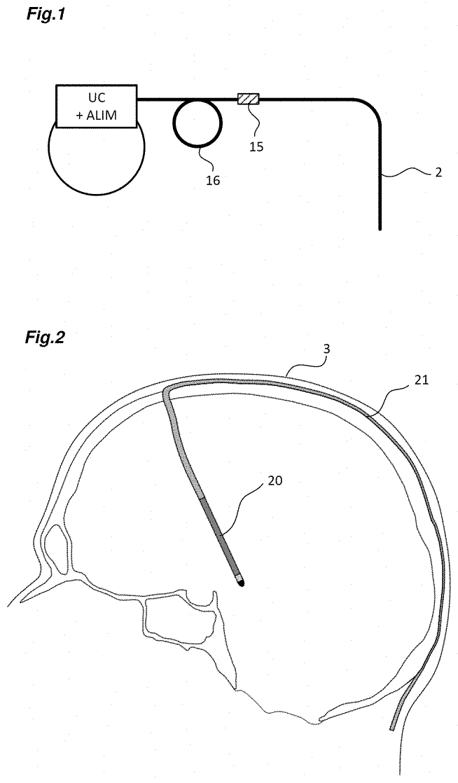

With reference to FIGS. 1 and 2, the cooling device of the invention thus mainly comprises:
a probe 2 intended to be at least partially implanted in the body of the patient, in the interior of the skull 3;
a central power and control unit ALIM, UC that is advantageously placed on the exterior of the skull of the patient, and to which the probe 2 is electrically connected.

The device 1 may comprise electrical connecting means allowing the probe 2 to be connected directly to the central unit via its proximal end, or indirectly via a connector 15 and an extension 16.

At the distal end, the probe 2 advantageously has an atraumatic shape (for example an oblong or spherical shape—see below).

Generally, the probe 2 has the following elements and functions:
one portion of the probe making contact with the brain and allowing the cooling;
a thermodynamic system allowing the natural direction of the heat transfer to be inverted;
a device for transporting heat from the region to be cooled to another region of the brain;
a thermally separating device between the cold portion and the hot portion of the device;
a heat-distributing device allowing the creation of a hotspot to be avoided;
a device allowing the probe to be powered while guaranteeing a supple mechanical link to the cranium.

These various elements and functions are embodied in the probe 2 in the way described below.

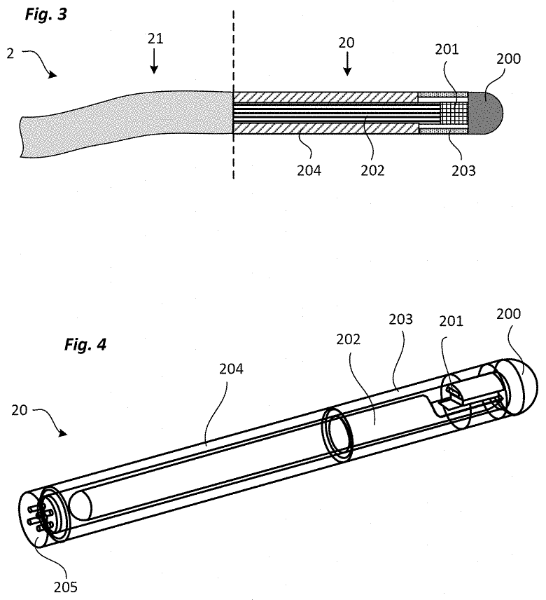

With reference to FIGS. 2 and 3, the probe 2 takes the form of an elongate rod that is developed about a longitudinal axis, called the main axis, between an end referred to as the distal end, which bears a cooling member, and an end referred to as the proximal end, which for its part may be connected to a processing and control unit UC. The probe is advantageously composed of two separate assemblies 20, 21 that are mechanically and electrically connectable, a rigid first assembly 20 that bears the cooling member and a second assembly 21 that allows the probe 2 to achieve a supple mechanical link to the cranium. The mechanical link to the cranium must allow the probe 2 to be held in place while limiting the risks of lesion. Supple power, control and measurement cables of the implant will possibly be integrated into the interior of this mechanical link. This link will advantageously have a central channel allowing a rigid stylus to be inserted with a view to facilitating surgical placement. This second assembly may take the form of a supple, single-channel or multichannel tube, made of silicone or polyurethane for example.

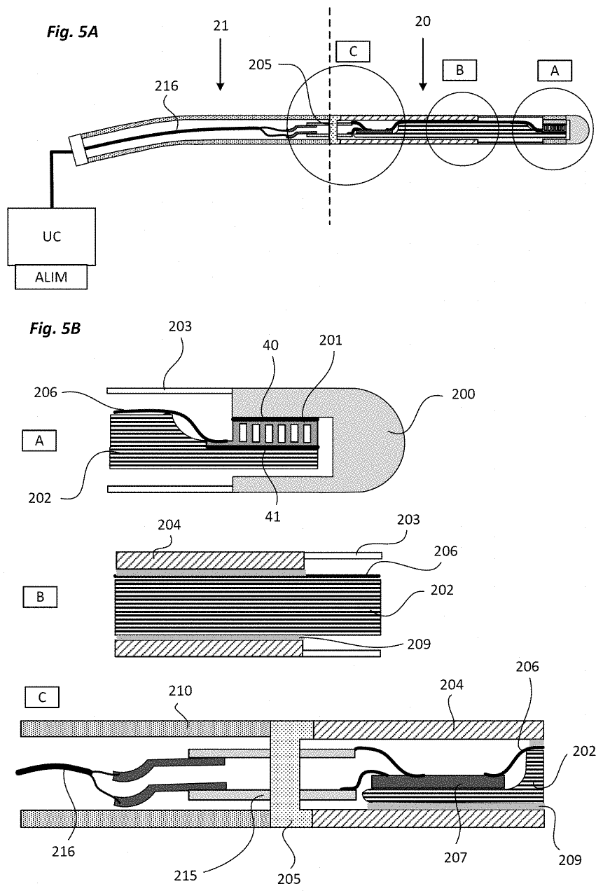

With reference to FIG. 4 and FIGS. 5A and 5B, the first assembly 20 of the probe 2 is described below, from its distal end to its proximal end.

The first assembly 20 of the probe therefore bears at its distal end a cooling member. This cooling member is advantageously a finger 200 (also called the cold finger) intended to be cooled and to make contact with the brain tissue to be cooled. Being intended to make contact with the brain, said member will necessarily be made of a biocompatible material.

The dimensions and shape of the cooling finger 200 define the cooled region and the power required to achieve this cooling. With a cold finger 200 of large size, the cooled region will be larger, but the medical risks also. It is therefore a question of defining the optimum compromise.

The cold finger 200 must thus meet the following constraints:
it must allow the largest possible volume of brain in the region of interest to be cooled,
it must be of limited bulk,
it must be able to be fitted simply, avoiding any risks of lesion.

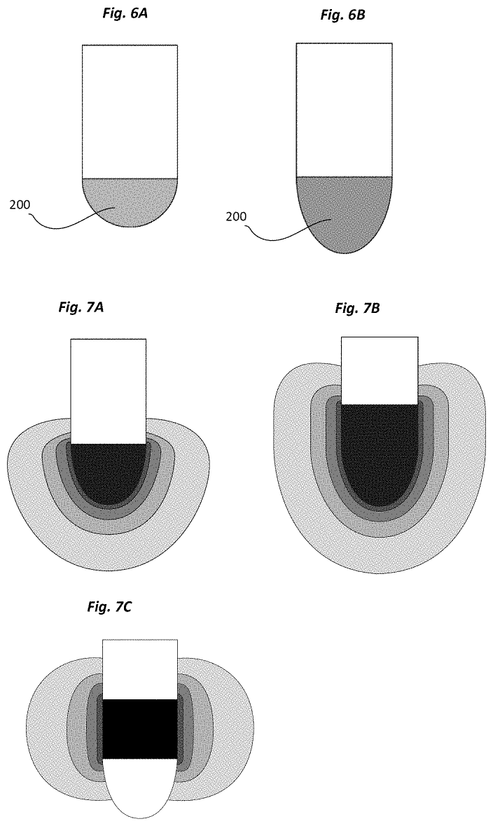

Thus, it must have a sufficient area of contact and a rounded or oblong final shape without any sharp edges. FIGS. 6A and 6B show two examples of embodiments of the cold finger 200. It should be noted that the use of a relatively pronounced oblong allows the type of target that it is desired to cool to be adapted to.

With reference to FIGS. 7A, 7B and 7C, three thermal simulations are thus presented.

In FIG. 7A, it is a question of thermal simulation with a probe of 3 mm diameter and a finger 200 of oblong shape.

If required, in order to increase the area of contact with the brain, it is possible to extend the hemisphere of the cold finger with a relatively long cylindrical portion, as illustrated in FIG. 7B.

In FIG. 7C, it is a question of thermal simulation with a probe of 3 mm diameter and a finger 200 of oblong shape for the purpose of achieving annular cooling.

Non-limitingly, the cold finger 200 will possibly be made of a material such as aluminium nitride, silicon, SiC or a platinum-iridium alloy, which are very good thermal conductors and biocompatible. Other metals such as titanium or a stainless steel may also be suitable to a lesser extent.

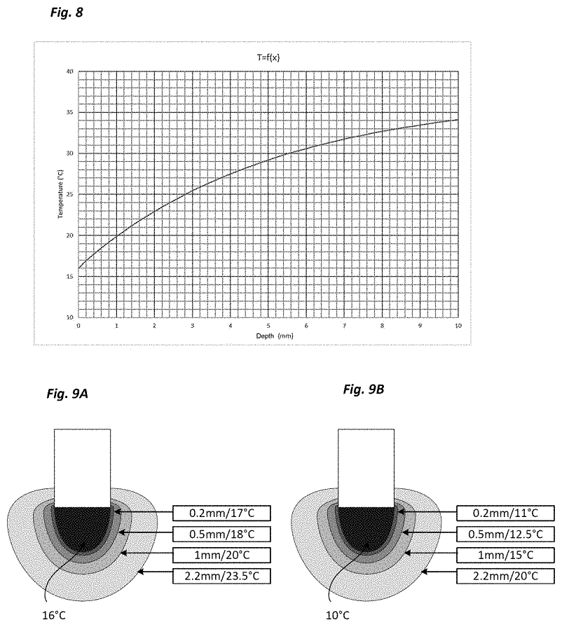

FIG. 8 shows a curve illustrating the temperature gradient on the periphery of a cold finger having a shape such as that shown in FIG. 7A, when said finger is at a temperature of 16° C. FIG. 9A illustrates this temperature gradient around the cold finger at 16° C. FIG. 9B for its part shows the temperature gradient about the same cold finger when the latter is at a temperature of 10° C.

The probe 2 comprises a thermoelectric cooling module 201. This thermoelectric cooling module 201 may be a Peltier-effect module, and more precisely a micro-Peltier module, in order to be suitable for the dimensions of the probe. The thermoelectric cooling module 201 is intended to convert an electrical current into a temperature difference, and the Peltier effect is a thermoelectric effect consisting in a physical phenomenon of movement of heat in the presence of an electrical current.

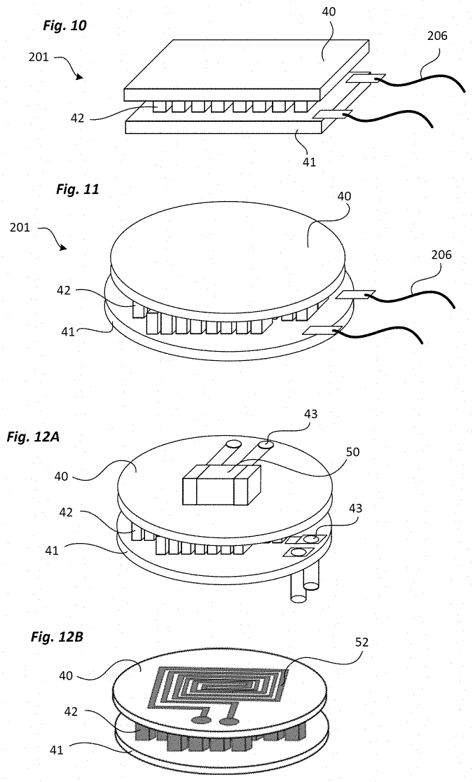

With reference to FIGS. 10 and 11, a thermoelectric cooling module is more precisely a cell manufactured with asymmetric semiconductor components 42. They are connected thermally in parallel and electrically in series between a plate 40 called the cold plate and a plate 41 called the hot plate. These two plates 40, 41 are generally made of ceramic. The semiconductors are p- and n-type. The cold plate is cooled via the absorption of energy due to the passage of electrons from one semiconductor to the next. The hot plate 41 collects the thermal energy captured from the cold plate 40. It is therefore vital to remove this heat so that it does not once again heat the cold plate.

Since the probe 2 has a diameter smaller than or equal to 3 mm, the dimensional constraints are tight. It is thus essential to correctly define the power level required for correct operation of the probe. To this end, the following requirements specific to the use of the device are used as starting point:
a probe 2 of 1 to 3 mm diameter;
a minimum cold-finger temperature of 4° C., in order not to cause tissue damage;
a cold finger 200 that reaches, for example, a temperature of 16° C. in less than 12 seconds for example.

After simulations, the cooling power of our thermodynamic system may be chosen comprised between 0.1 W and 0.4 W, neglecting losses. These thermal losses will mainly be related to the contact created by the packaging between the hot region and the cold region of our system.

FIG. 15 illustrates this choice. In this graph, the power required to keep the finger at 4° C. as a function of time has been drawn for a probe of 3 mm diameter and a stainless-steel oblong cold finger of 2.5 mm length.

To maintain a steady state, a minimum cooling power of 0.37 W is required, neglecting losses.

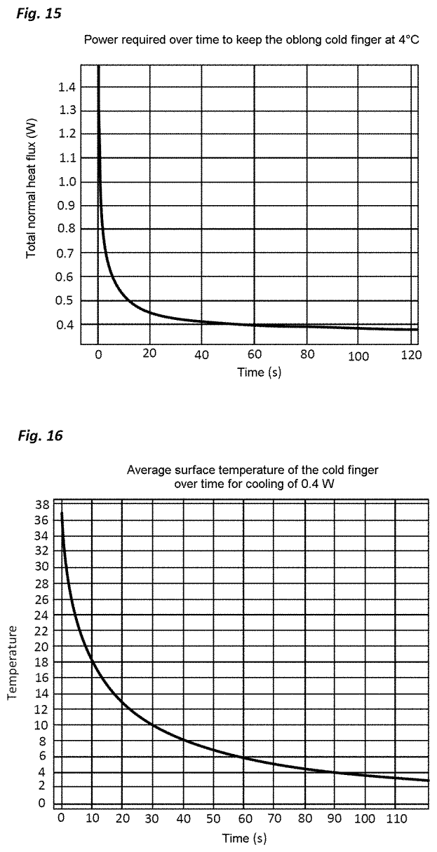

If a simulation is performed with the same device and a thermodynamic system of a power of 0.4 W, the variation in the temperature of the cold finger over time after the system is started up is obtained. This variation is shown in the graph of FIG. 16. It may notably be seen that, with a cooling power of 0.4 W, it is possible to obtain an average cold-finger temperature lower than 16° C. in less than 12 seconds.

Non-limitingly, the cooling power of the thermodynamic system is comprised between 0.1 W and 0.4 W, neglecting losses.

Non-limitingly, the micro-Peltier module 201 may be that sold by TEC MICROSYSTEMS under the reference 1MD02-010-03ANt.

The thermoelectric cooling module 201 allows heat to be drawn from the brain via the cold finger 200. To remove the heat and transport it to a region further away, the probe 2 next comprises a heat-transporting device 202.

This heat-transporting device 202 has a high thermal conductivity, for example one which is higher than 1000 W/m/K.

This function may for example be performed using a micro-heat pipe made of a metal material such as copper or of silica, sapphire or silicon to decrease artefacts during MRI imaging.

As a variant, it may also be an element made of pyrolytic graphite.

As shown in FIG. 5B, the heat-transporting device 202 comprises a first end that makes contact with the hot plate 41 of the thermoelectric cooling module 201, in order to collect as best as possible the heat to be removed.

Non-limitingly, in a first variant embodiment, the heat-transporting device may comprise, at this first end, a beak defining a contact surface arranged parallel to the axis of the probe, against which is placed the hot plate 41 of the thermoelectric cooling module 201. The thermoelectric cooling module 201 thus has its hot plate 41 against said contact surface and its cold plate 40 arranged parallel to its hot plate. The cold finger 200 may thus comprise an extension allowing it to press against the cold plate 40 of the module and thus to ensure heat transfer from the module to the cold finger 200. The thermoelectric cooling module 201 for example has the configuration shown in FIG. 10, with plates 40, 41 of rectangular shape.

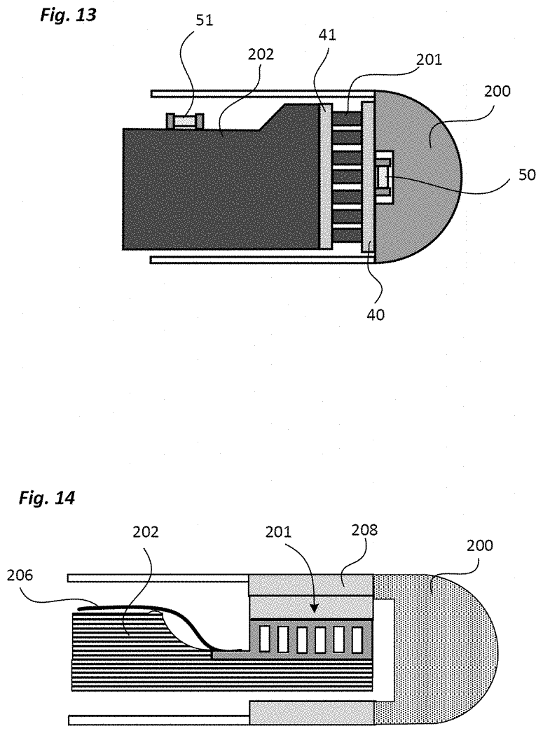

In a second variant embodiment illustrated in FIG. 13, the contact surface may be arranged perpendicular to the axis of the probe 2. The thermoelectric cooling module 201 therefore has its hot plate 41 arranged transversely to the axis of the probe against the contact surface and its cold plate 40 parallel, against which the cold finger 200 is pressed. This solution allows the available power and thus the temperature gradient to be increased.

In the latter case, the thermoelectric cooling module 201 then for example has the configuration shown in FIG. 11, with disc-shaped plates 40, 41, with a view to being integrated transversely into the probe 2.

With reference to FIG. 12A, this ad hoc Peltier module may also incorporate through-contacts 43 in the hot and cold plates 40, 41, in order to facilitate its incorporation into the probe 2.

In FIG. 12A an SMD temperature probe 50 is present on the cold face 40. As shown in FIG. 12B, this temperature probe may advantageously be replaced by a screen-printed probe 52, a PT100 probe for example. This type of screen-printed probe may be produced on the external faces of the plates 40 and 41.

The heat-transporting device 202 extends along the main axis of the probe over a non-zero determined length to the proximal side of the probe 2.

To avoid heat losses in proximity to the region to be cooled and therefore to better remove the heat by virtue of the heat-transporting device 202, the probe 2 comprises a thermally separating device. It is advantageously a question of a thermally separating jacket, called the insulating jacket 203, arranged around a first portion of the length of the heat-transporting device 202. The role of this insulating jacket 203 is to thermally separate the cold-finger portion and the heat-distributing portion. It makes no thermal contact with the heat pipe or the Peltier module (see below). As it is a question of cooling a region of the brain, any transfer of heat to this region specifically decreases the effectiveness of the system and it is therefore essential to adequately separate the cold region from the hot region. Moreover, the length of the insulating jacket 203 must be as small as possible in order to consecrate a maximum length to the thermal distribution. Since the cooling power of the thermoelectric cooling module 201 is comprised between 100 W and 400 mW, the insulating jacket 203 will possibly extend over a length of at least 6 mm and have the smallest possible thickness, 0.25 mm for example. These dimensional data are to be considered to be non-limiting and will of course vary depending on the cooling power of the thermoelectric cooling module 201.

Non-limitingly, this insulating jacket 203 may take the form of a silica or zirconium ring.

The combination of modules 200, 203, 204 and 205 is hermetically assembled by laser welding or brazing.

According to the invention, in a second portion of the length of the heat-transporting device 202, the probe 2 comprises a heat-distributing device (also called the heat “distributor” below). This device is intended to allow the heat transported by the heat-transporting device to be released at a sufficient distance from the region to be cooled, using the arterial perfusion of the brain. This device thus acts as a heat exchanger with a view to removing the heat absorbed by the cold finger. Its objective is therefore to cede thermal energy to the surrounding brain, without creating a lesion.

With reference to FIGS. 4 and 5B, this heat-distributing device takes the form of a jacket 204 of good thermal conductivity, called the conductive jacket 204, which is arranged around a second portion of the heat-transporting device 202. This conductive jacket 204 extends the insulating jacket 203 forming the thermal separation, in the direction of its proximal end. It therefore of course has a thermal conductivity higher than that of the insulating jacket 203. It advantageously makes contact with the second portion of the heat-transporting device 202. By way of example, it may be assembled therewith by means of a thermally conductive adhesive 209.

As regards the optimum length of this conductive jacket 204, it may be computed using the equation of heat transfer in a heat exchanger:

P=h×S×DT

with
P the power to be ceded to the brain, corresponding to the power extracted by the cold finger divided by the efficiency of the thermoelectric cooling module;
h the total exchange coefficient;
S the area of the exchanger-brain interface;
DT the temperature difference between the exchanger and the brain.

The only variable it is possible to control is the area of the exchanger. The diameter of the probe 2 being set, the optimal length of the exchanger (the conductive jacket) is sought with a view to avoiding overheating of the brain, which could lead to lesions. It is for example chosen to limit maximum heating of the brain to 2° C. with respect to equilibrium.

Non-limitingly, it is possible to make provision for a length at least equal to 50 mm of exchanger for a diameter of 2.5 to 3 mm, this allowing overheating at the end of 3 minutes of operation with a power of 0.35 W to be avoided.

Non-limitingly, this conductive jacket 204 may be made of a material such as a metal, titanium, allowing the creation of hotspots to be avoided. It may also be made of a material of sapphire, aluminium nitride or alumina type in order to improve MRI compatibility. To make thermal contact between 202 and 204, an epoxy resin (209), polyurethane or silicone that is filled (to improve its thermal conductivity) may for example be injected into the internal channel of the probe 2, advantageously solely around the second portion of the heat-transporting device 202, in order to fill the space between the latter and the conductive jacket 204.

At its proximal end, the first assembly 20 of the probe 2 may provide an electrical feed-through 205 to which power conductors of the thermoelectric cooling module 201 and other connection points allowing the transfer of data (temperature, measurement electrodes) are joined. It allows the first assembly 20 of the probe to be connected to the second assembly 21 of the probe.

The conductors 206 may be electrical tracks of a flexible electrical circuit, which is for example made of polyimide, or they may be arranged directly on the heat-transporting device 202. A contact-redistribution board 207 may be arranged to hold all the electrical components for control, signal conditioning, electrical safety and for connecting the conductors to the respective pins of the electrical feed-through. Of course, since the electrical connection and linking solutions do not form part of the subject matter of the invention, any other solution could be envisioned.

The external envelope of the device, which envelope is formed by that of the cold finger 200, of the insulating jacket 203, of the conductive jacket 204 and the electrical feed-through 205, meets the conditions of biocompatibility and said jacket and feed-through are assembled together, for example using technologies such as vacuum and/or laser brazing.

This second assembly 21 of the probe may take the form of a supple, single-channel or multichannel tube 210, of biocompatible silicone or polyurethane for example. The conductors 206 are extended in the interior of the tube by a supple cable 216 in order to reach the processing and control unit UC. The cables 216 may be connected to the contacts 215 by spot soldering or laser welding before the protective tube 210 is assembled by adhesive bonding.

Non-limitingly, the control unit UC comprises at least one microprocessor and means for storing in memory. It is intended to execute software instructions representative of a sequence of treatment of the pathology by the device. It notably comprises means for controlling the cooling device. It also comprises one or more communication interfaces intended to communicate with various entities, notably detecting means. The communication links will possibly be wired or wireless.

The cooling device comprises an electrical power source ALIM, for powering the probe and the other elements of the device. Non-limitingly, the electrical power source ALIM may be a rechargeable battery. The battery and the control unit UC are advantageously incorporated into a hermetic casing equipped with a connector and are located on the exterior of the skull 3.

Various additions may be made to the probe of the invention. We will give below a few examples of complementary solutions that it is possible to incorporate into this probe.

A solution for monitoring the temperature at various points on the probe may be incorporated. FIG. 13 gives a few examples of locations of temperature sensors on the probe. This is of course applicable to the various variants of the probe 2, irrespectively of whether the thermoelectric cooling module 201 is axial (FIG. 5A) or transversal (FIG. 13).

It is thus possible to place a first probe 50 (a thermistor for example) next to the cold finger, on the cold plate 40 of the thermoelectric cooling module. A second probe 51 (a thermistor for example) may be placed in the hot region in contact with the plate 41, and another in the heat-exchange region of the conductive jacket 204. It will be noted that these probes may also take the form of a screen-printed probe 52, as shown in FIG. 12B.

A thermostatic relay may also be employed in the hot portion 204, ideally on the circuit board 207, to cut power in case of failure of the regulating system and thus guarantee the safety of the patient. With respect to temperature, the safety of the cold portion 200 is ensured by limiting the power of the system.

The temperature data measured by the various sensors are advantageously sent to the control unit UC with a view to controlling, in real-time, the cooling level applied by the device of the invention and to regulating it, if necessary, by executing a temperature regulation loop. The temperature sensors may be any type of temperature sensor (thermistor, PT100, thermocouples), connected via a wired link to said control unit UC. In the case of a wired link, the wire may pass through the probe 2 in a direction parallel to its axis.

Detecting means may be integrated in parallel to detect the appearance of the pathology to be treated. In the case of an epileptic fit, these detecting means are implanted into the skull in order to detect a targeted region. When the beginnings of a fit are detected, the detecting means may send a signal to the control unit UC. The control unit UC supplies power to the probe 2. The cooling device of the invention is controlled to generate cooling suitable for the treated pathology. The detecting means may comprise EEG electrodes or SEEG electrodes (intracranial stereoelectroencephalography). They may also be formed by an assembly of metal rings or localized deposition directly on the cold finger in order to be as close as possible to the region to be treated.

Temperature regulation may be achieved by virtue of the temperature sensor placed at the end of the probe, depending on a temperature setpoint. The temperature setpoint may be programmed into the control unit UC. If it is a question of an epileptic fit, the magnitude of the cooling and its duration of application will preferably be related to the level of severity of the fit, which will be measured by the detecting means. In this context, given that an epileptic fit results in a small amount of heating, the various temperature sensors proposed above could allow false positives detected by the SEEG electrodes to be eliminated and thus detection to be improved.

The control unit UC of the device may incorporate a current rectifier so as to limit the risks inherent to leakage currents and thus allow a sinusoidal or pulsed AC supply current that generates an average current of zero to be used.

The implant may be fitted using a peelable guide or a rigid mandrel. The latter will have to be removed once the device is in place.

With reference to FIG. 14, it may also be envisioned to make the finger 200 located at the end of the probe of a material that is not thermally conductive, of silica or of zirconium for example, and to insert a cooling ring 208 directly around the thermoelectric cooling module 201. Cooling is thus no longer achieved axially but rather radially. The other particularities of the probe 2 remain the same. FIG. 7C, which was mentioned above, moreover illustrates the temperature gradient obtained around this cooling ring.

The solution of the invention thus has many advantages, among which:
a thermal architecture that is optimized, as regards the delivery of cold to the end of the probe and the removal of heat;
a probe of a size and shape that are suitable for long-term implantation, allowing mechanical lesions to be avoided while guaranteeing a sufficient robustness and allowing the targeted region to be cooled effectively, with optimal cooling;
a solution that has mechanical properties suitable for long-term implantation at depth, notably by virtue of its two-part structure (rigid assembly and supple assembly);
a solution that uses materials that are suitable for long-term implantation at depth, and that are all biocompatible.

1. A probe to be at least partially implanted into a living being, to locally cooling at least one region of the living being, said probe having a shape that is elongate along a longitudinal axis between a first end, referred to as the proximal end, and a second end, referred to as the distal end, said distal end being intended to make contact with the region to be cooled, wherein said probe has a first assembly comprising:
a cooling member present at its distal end, said member being intended to make contact with the region to be cooled,
a thermoelectric cooling module comprising a cold region making contact with the cooling member and a hot region,
a device for transporting heat having a first portion making contact with the hot region of the thermoelectric cooling module and a second portion extending the first portion towards the proximal end of the probe,
a thermally separating first jacket arranged around the first portion of said heat-transporting device,
a heat-dissipating second jacket arranged in contact with the second portion of said heat-transporting device, said heat-dissipating second jacket having a thermal conductivity higher than that of the thermally separating jacket,
electrical connecting means arranged along the probe with a view to powering the thermoelectric cooling module.

2. The probe according to claim 1, wherein the first assembly is of unitary construction and has a rigid configuration, and wherein the probe comprises a second assembly made of a more supple material extending said first assembly towards its proximal end.

3. The probe according to claim 2, wherein electrical connecting means are arranged in the first assembly and in the second assembly.

4. The probe according to claim 2, wherein the second assembly takes the form of a supple tube made of silicone or polyurethane.

5. The probe according to claim 1, wherein the heat-transporting device is a heat pipe or a heat-transferring system made of pyrolytic graphite.

6. The probe according to claim 1, wherein the thermally separating first jacket is made of silica or zirconium.

7. The probe according to claim 1, wherein the heat-dissipating second jacket is made of sapphire or aluminium nitride.

8. The probe according to claim 1, wherein the thermoelectric cooling module is a Peltier-effect module having a face called the cold face and a face called the hot face.

9. The probe according to claim 8, wherein the Peltier-effect module is arranged with the cold face and the hot face parallel to said longitudinal axis.

10. The probe according to claim 8, wherein the Peltier-effect module is arranged with the cold face and the hot face arranged transversely to said longitudinal axis.

11. The probe according to claim 1, wherein a temperature probe is arranged in contact with the cooling member.

12. A cooling device comprising a control unit and a probe to be at least partially implanted in a living being, with a view to locally cooling at least one region of the living being, wherein said probe is such as defined in claim 1.
